(19) United States
(12) Patent Application Publication
(10) Pub. No.: US 2010/0125323 A1
(43) Pub. Date: May 20, 2010

(54) Coil Stent Delivery System and Method of Use

(75) Inventors: Joseph Berglund, Santa Rosa, CA (US); Matthew J. Birdsall, Santa Rosa, CA (US); Lesley Crookall, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/271,465

(22) Filed: Nov. 14, 2008

(51) Int. Cl. A61F 2/06

(52) U.S. Cl. 623/1.11; 623/1.15; 623/1.22

(57) ABSTRACT

The coil stent delivery system and method of use includes a stent delivery system including a coil stent; a housing, the housing having a receiver defining a receiver chamber and a sheath defining a sheath lumen, the receiver chamber being in communication with the sheath lumen; and a screw assembly, the screw assembly having a shaft, a helical screw disposed about a distal portion of the shaft, and a drive operably coupled to the shaft. The shaft is disposed in the receiver chamber and the sheath lumen, the helical screw is disposed in the sheath, and the coil stent is disposed about the shaft in the receiver chamber and engages the helical screw. Rotation of the drive moves the coil stent through the sheath lumen.

### TECHNICAL FIELD

The technical field of this disclosure is delivery systems for medical implant devices, particularly, delivery systems for stents.

### BACKGROUND OF THE INVENTION

Stents are generally cylindrical shaped devices that are radially expandable to hold open a segment of a blood vessel or other anatomical lumen after implantation into the body lumen. Stents have been developed with coatings to deliver drugs or other therapeutic agents.

Stents are used in conjunction with balloon catheters in a variety of medical therapeutic applications including intravascular angioplasty. For example, a balloon catheter device is inflated during PTCA (percutaneous transluminal coronary angioplasty) to dilate a stenotic blood vessel. The stenosis may be the result of a lesion such as a plaque or thrombus. After inflation, the pressurized balloon exerts a compressive force on the lesion thereby increasing the inner diameter of the affected vessel. The increased interior vessel diameter facilitates improved blood flow. Soon after the procedure, however, a significant proportion of treated vessels re-narrow.

To prevent restenosis, short flexible cylinders, or stents, constructed of metal or various polymers are implanted within the vessel to maintain lumen size. The stents acts as a scaffold to support the lumen in an open position. Various configurations of stents include a cylindrical tube defined by a mesh, interconnected stents or like segments. Some exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz and U.S. Pat. No. 5,421,955 to Lau. Balloon-expandable stents are mounted on a collapsed balloon at a diameter smaller than when the stents are deployed. Stents can also be self-expanding, growing to a final diameter when deployed without mechanical assistance from a balloon or like device.

One approach has been to develop coil stents in which the stent is a continuous helical coil. Coil stents can provide advantages over conventional stents in post-deployment flexibility and strength. Unfortunately, the relationship between coil diameter and the number of coil turns in crimped and deployed configurations complicates delivery of the coiled stents: the pitch change from the crimped to deployed configurations requires the coil stent to unwrap and unfurl. Coil stents exhibit a large degree of foreshortening, which requires the coil stent to be much longer in the crimped configuration than in the deployed configuration. This large length causes problems with pushability and column strength when the coil stent is advanced to the deployment site through a catheter.

Another problem with coil stents made of polymers, such as bioabsorbable polymers, is their tendency to creep and take a permanent set when held in constrained configurations for extended periods. This precludes pre-loading the polymer coil stents in a crimped configuration at the point of manufacture, and instead requires they be loaded into delivery systems just before implantation at the catheterization laboratory. This increases cost of procedures due to the need of trained staff to load the coil stent and increases the chance of mistakes.

It would be desirable to have a coil stent delivery system and method of use that would overcome the above disadvantages.

### SUMMARY OF THE INVENTION

One aspect of the present invention provides a stent delivery system including a coil stent; a housing, the housing having a receiver defining a receiver chamber and a sheath defining a sheath lumen, the receiver chamber being in communication with the sheath lumen; and a screw assembly, the screw assembly having a shaft, a helical screw disposed about a distal portion of the shaft, and a drive operably coupled to the shaft. The shaft is disposed in the receiver chamber and the sheath lumen, the helical screw is disposed in the sheath, and the coil stent is disposed about the shaft in the receiver chamber and engages the helical screw. Rotation of the drive moves the coil stent through the sheath lumen.

Another aspect of the present invention provides a delivery system for a coil stent including a housing, the housing having a receiver defining a receiver chamber and a sheath defining a sheath lumen, the receiver chamber being in communication with the sheath lumen; and a screw assembly, the screw assembly having a shaft, a helical screw disposed about a distal portion of the shaft, and a drive operably coupled to the shaft. The shaft is disposed in the receiver chamber and the sheath lumen, the helical screw is disposed in the sheath, and the coil stent is disposed about the shaft in the receiver chamber and engages the helical screw. Rotation of the drive moves the coil stent through the sheath lumen.

Another aspect of the present invention provides a method of delivering a coil stent including providing a coil stent delivery system, the coil stent delivery system having a sheath defining a sheath lumen and a shaft with a helical screw disposed about the shaft in the sheath lumen; advancing a distal end of the sheath to a deployment site; engaging the coil stent with the helical screw; and rotating the shaft to rotate the helical screw and urge the coil stent through the sheath toward the deployment site.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1 is an exploded side view of a coil stent delivery system made in accordance with the present invention.

FIG. 2 is a cross section view of a coil stent delivery system made in accordance with the present invention.

FIGS. 3A-3D are cross section views of deployment of a coil stent with a coil stent delivery system made in accordance with the present invention.

FIGS. 4A & 4B are cross section views of another deployment of a coil stent with a coil stent delivery system made in accordance with the present invention.

FIGS. 5A & 5B are cross section views of deployment of a coil stent with other embodiments of a coil stent delivery system made in accordance with the present invention.

FIG. 6 is a cross section view of yet another embodiment of a coil stent delivery system made in accordance with the present invention.

FIG. 7 is a cross section view of yet another embodiment of a coil stent delivery system made in accordance with the present invention.

FIG. 8 is a flow chart for a method of delivering a coil stent in accordance with the present invention.

### DETAILED DESCRIPTION

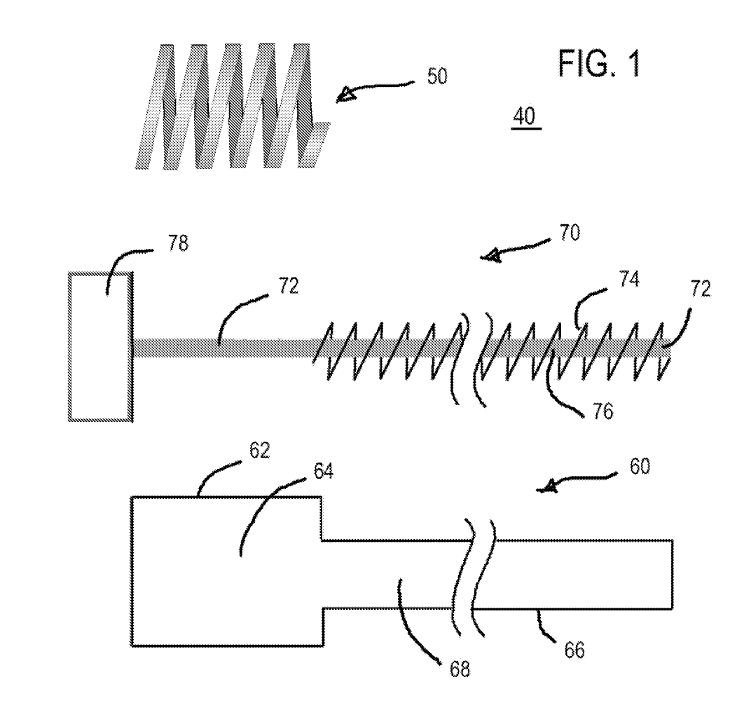

FIG. 1 is an exploded cross section view of a coil stent delivery system made in accordance with the present invention. The stent delivery system 40 includes a coil stent 50, a housing 60, and a screw assembly 70. The housing 60 has a receiver 62, which defines a receiver chamber 64, and a sheath 66, which defines a sheath lumen 68. The receiver chamber 64 is in communication with the sheath lumen 68. The screw assembly 70 has a shaft 72, a helical screw 74 disposed about a distal portion 76 of the shaft 72, and a drive 78 operably coupled to the shaft 72. When the stent delivery system 40 is assembled, the shaft 72 is disposed in the receiver chamber 64 and the sheath lumen 68, the helical screw 74 is disposed in the sheath 66, and the coil stent 50 is initially disposed about the shaft 72 in the receiver chamber 64 and engages the helical screw 74. The drive 78 can rotate the shaft 72, which rotates the helical screw 74, to move the coil stent 50 through the sheath lumen 68. As used herein, distal and proximal are from the viewpoint of the operator of the stent delivery system 40, e.g., the receiver 62 is towards the proximal end of the housing 60 and the sheath 66 is towards the distal end.

The coil stent 50 can be any generally helical shaped stent. The coil stent 50 has a relaxed diameter when unconstrained and a delivery diameter when constrained within the sheath 66 during delivery. The diameter of the coil stent 50 as defined herein is the diameter across the central axis of the stent coil. The diameter of the receiver chamber 64 can be greater than or equal to the relaxed diameter of the coil stent 50 so the coil stent 50 is maintained in a relaxed configuration. The diameter of the sheath lumen 68 is the delivery diameter. In one embodiment, the coil stent 50 is sealed in the receiver chamber 64 at the time of manufacture to avoid having to load the coil stent 50 into the housing 60 at the catheterization laboratory. The coil stent 50 can have a cross section across the coil at the perimeter which is a circle, rectangle, ellipse, or any other cross section as desired for a particular application. The coil stent 50 can be the final length to be deployed at the deployment site, or can be a coil stent blank, which is longer than the desired final length and is cut to the desired length at the deployment site.

The coil stent 50 can be made of one or more biocompatible materials suitable for a particular application. In one embodiment, the coil stent 50 is bioabsorbable and can be made of a bioabsorbable material such as homopolymers and copolymers (including random and block polymers) of D-lactide, L-lactide, DL-lactide, caprolactone, trimethylenecarbonate, glycolide, caprolactone derivatives, P-Dioxanone, hydrolysable urethanes, and combinations thereof. Polyethylene oxide can be part of the polymer chain. Another exemplary material is degradable polyurethane. In one embodiment, the coil stent 50 is non-bioabsorbable and can be made of a non-bioabsorbable material such as homopolymers and copolymers of ethylene, propylene, amides, esters, acrylates, carbonates, imides, styrenes, non-hydrolysable urethanes, combinations thereof and the like. In another embodiment, at least an end portion of the coil stent 50 is made of a super-elastic material, such as nitinol or the like. In an exemplary coil stent 50 having an end portion of super-elastic material and a middle portion of a polymeric material, the super-elastic material end portion aids in deployment of the coil stent 50 since the super-elastic material is radially stronger than the polymeric material. One or both end portions of the coil stent 50 can be made of the super-elastic material.

In one embodiment, the coil stent 50 can be capable of carrying a coating, such as a polymer coating carrying one or more therapeutic agents, such as anti-inflammatory agents or anti-proliferative agents. In another embodiment, the coil stent 50 can include one or more therapeutic agents within the stent material.

The housing 60 is adapted to receive coil stent 50 and the screw assembly 70 in the receiver chamber 64 of the receiver 62 and the sheath lumen 68 of the sheath 66. The screw assembly 70 is free to rotate within the housing 60. The rotation of the screw assembly 70 moves the coil stent 50 axially from the receiver chamber 64 through the distal end of the sheath 66, which is open, and out of the sheath 66 to the deployment site. In one embodiment, the sheath 66 can be rotated and moved axially relative to the screw assembly 70 without moving the screw assembly 70, to assist in opening, sizing, and placement of the coil stent 50 at the deployment site. The sheath 66 can be flexible to allow advancement through a tortuous vasculature to a remote deployment site. The inner walls of the sheath 66 can be lubricated or have a lubricating coating to ease the passage of the coil stent 50 through the sheath 66. In one embodiment, the sheath 66 can include a cutter, such as a radio frequency (RF) cutter, at the distal end to cut the coil stent 50 to the desired length at the deployment site.

The screw assembly 70 has a shaft 72, a helical screw 74 disposed about a distal portion 76 of the shaft 72, and a drive 78 operably coupled to the shaft 72. The helical screw 74 acts like an Archimedes screw or a screw conveyor to move the coil stent 50 through the sheath 66 when the shaft 72 rotates the helical screw 74. In one embodiment, the helical screw 74 is a continuous screw. In another embodiment, the helical screw 74 is a series of separate screw blades. In one embodiment, the surface of helical screw 74 in contact with the coil stent 50 is covered with a low-tack adhesive to assist in moving the coil stent 50 through the sheath 66.

The shaft 72 can include a central shaft lumen. In one embodiment, the shaft lumen is operable to receive a guidewire. A guide wire is advanced through the vasculature to a deployment site and the stent delivery system 40 is advance over the guide wire in the shaft lumen of the shaft 72. In another embodiment, coolant is disposed in the shaft lumen to cool the coil stent 50, such as a coil stent made of a shape memory material, and prevent shape changes during deployment.

The shaft 72 can be flexible to allow advancement to a remote deployment site. In one embodiment, the shaft 72 can be a hypotube with grooves for increased flexibility. As defined herein, the grooves are cuts partially or completely through the thickness of the wall of the hypotube. The axial distribution of the grooves can be selected to tailor the flexibility of the shaft 72 as desired for a particular application. In one embodiment, the hypotube includes more grooves in a distal portion of the shaft 72 than in a proximal potion of the shaft 72, so that the distal portion of the shaft 72 is more flexible. In one example, a 20 centimeter distal portion of the shaft 72 is more flexible and a 70 centimeter proximal potion of the shaft 72 is less flexible.

The drive 78 operably coupled to drive the shaft 72 can be a manual drive, such as a handle, or a motor. The drive 78 can rotate in one direction to move the coil stent 50 in an axial direction toward the deployment site and in the opposite direction to move the coil stent 50 in an axial direction away from the deployment site. The reverse rotation allows retraction of a coil stent 50 that is partially deployed at the deployment site when the operator determines that the placement of the coil stent 50 at the deployment site is not as desired. In one embodiment, a rotation counter can be operably connected to the helical screw to count rotations and determine the axial position of the coil stent 50 in the sheath 66 from the number of rotations.

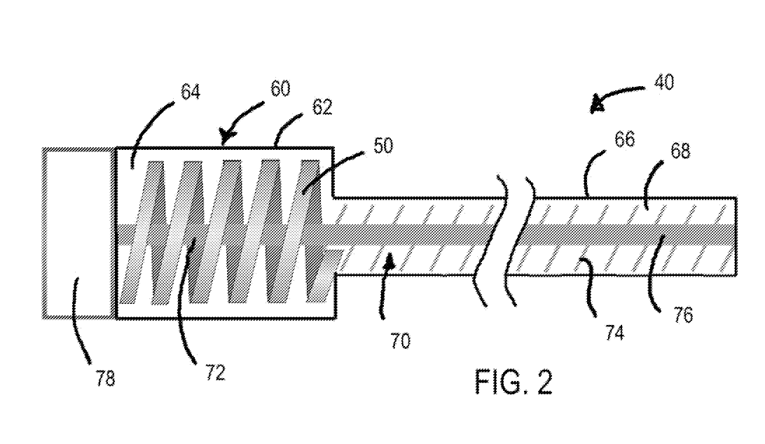

FIG. 2, in which like elements share like reference numbers with FIG. 1, is a cross section view of a coil stent delivery system made in accordance with the present invention. The shaft 72 is disposed in the receiver chamber 64 and the sheath lumen 68, the helical screw 74 is disposed in the sheath 66, and the coil stent 50 is disposed about the shaft 72 in the receiver chamber 64. The distal end of the coil stent 50 engages the helical screw 74. The helical screw 74 is shown schematically as a series of parallel lines along the shaft 72 for clarity of illustration. Rotation of the helical screw 74 moves the coil stent 50 axially through the sheath lumen 68. The diameter of the receiver chamber 64 is greater than or equal to the relaxed diameter of the coil stent 50 to avoid stressing the coil stent 50 when disposed in the receiver chamber 64. Since the coil stent 50 is not stressed, the coil stent 50 can be sealed in the receiver chamber 64 and the coil stent delivery system 40 delivered to the catheterization laboratory with the coil stent 50 pre-loaded.

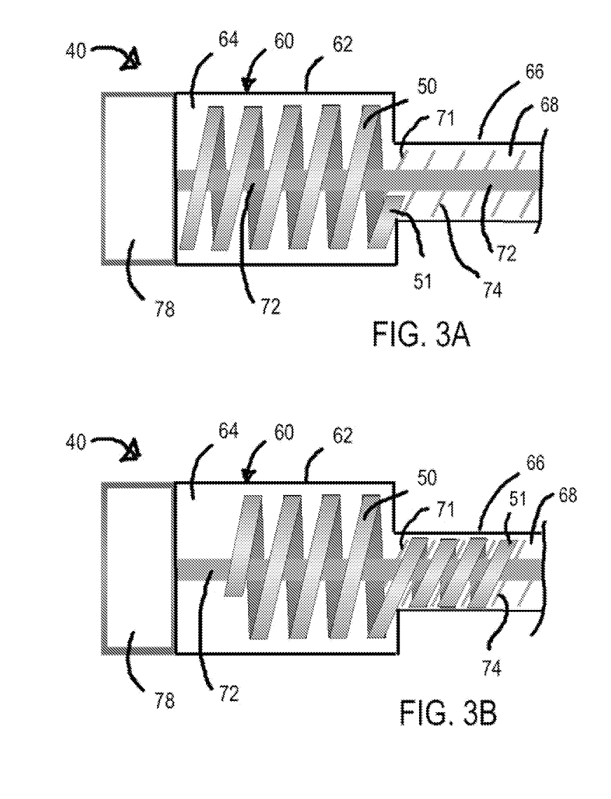

FIGS. 3A-3D, in which like elements share like reference numbers with each other and with FIG. 2, are cross section views of deployment of a coil stent with a coil stent delivery system made in accordance with the present invention. FIGS. 3A & 3B illustrate the transition of the coil stent from the receiver chamber to the sheath lumen at the proximal portion of the coil stent delivery system. FIGS. 3C & 3D illustrate the transition of the coil stent from the sheath lumen to the vessel lumen at the deployment site.

Referring to FIG. 3A, the distal tip 51 of the coil stent 50 is engaged with the proximal portion 71 of the helical screw 74. The coil stent 50 is at a relaxed diameter. The operator rotates the driver 78, which rotates the shaft 72 and the helical screw 74. Referring to FIG. 3B, the distal tip 51 of the stent coil 50 is drawn into the helical screw 74 and moves axially through the sheath lumen 68 toward the deployment site. The coil stent 50 is compressed to the delivery diameter by the sheath 66 to provide a small crossing profile. Further rotation of the driver 78 moves the coil stent 50 to the distal end of the sheath 66.

Referring to FIG. 3C, the distal tip 51 of the coil stent 50 emerges from the distal end 61 of the sheath 66 at the deployment site 82 in the vessel 80. The coil stent 50 expands from the delivery diameter in the sheath 66 to the deployment diameter in the vessel 80. Referring to FIG. 3D, the rotation of the helical screw 74 continues to move the coil stent 50 from the sheath 66 to the vessel 80 until the whole coil stent 50 is in the vessel 80 at the deployment site 82. In one embodiment, the sheath 66 and helical screw 74 are retracted as the coil stent 50 emerges from the sheath 66. In another embodiment, the sheath 66 and helical screw 74 are held in a fixed axial position relative to the vessel 80 as the coil stent 50 emerges from the sheath 66. The coil stent delivery system can be removed from the vasculature after the coil stent has been deployed.

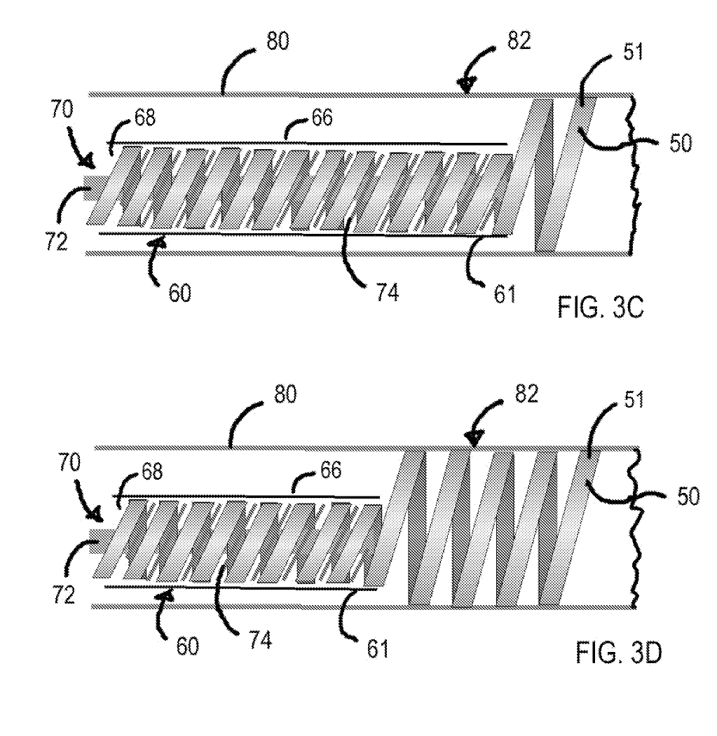

FIGS. 4A & 4B are cross section views of another deployment of a coil stent with a coil stent delivery system made in accordance with the present invention. FIGS. 4A & 4B show the transition of the coil stent from the sheath lumen to the vessel lumen at the deployment site.

Referring to FIG. 4A, the distal tip 51 of the coil stent 50 emerges from the distal end 61 of the sheath 66 at the deployment site 82 in the vessel 80. The coil stent 50 expands from the delivery diameter in the sheath 66 to the deployment diameter in the vessel 80. Referring to FIG. 4B, the rotation of the helical screw 74 continues to move the coil stent 50 from the sheath 66 to the vessel 80 until the whole coil stent 50 is in the vessel 80 at the deployment site 82. In this example, the sheath 66 is retracted while the helical screw 74 is held in a fixed axial position relative to the vessel 80. The coil stent 50 expands to the deployment diameter in the vessel 80 as the coil stent 50 becomes free of the sheath 66. The coil stent delivery system can be removed from the vasculature after the coil stent has been deployed.

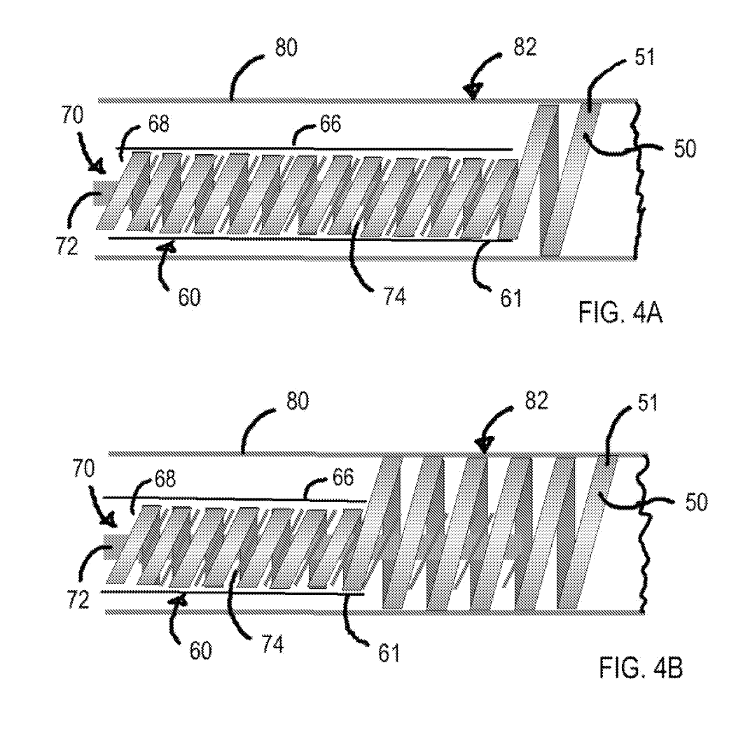

FIGS. 5A & 5B are cross section views of deployment of a coil stent with other embodiments of a coil stent delivery system made in accordance with the present invention. In this embodiment, the coil stent is cut to length at the deployment site.

Referring to FIG. 5A, the distal tip 51 of the coil stent 50 emerges from the distal end 61 of the sheath 66 at the deployment site 82 in the vessel 80. The coil stent 50 expands from the delivery diameter in the sheath 66 to the deployment diameter in the vessel 80. In this embodiment, the coil stent 50 is a coil stent blank, which is longer than the desired final length and is cut to the desired length at the deployment site. The sheath 66 includes a cutter 90, such as a radio frequency (RF) cutter, at the distal end 61 of the sheath 66. Once the desired length of the coil stent 50 is located at the deployment site 82, the cutter 90 is activated to cut the coil stent 50. Referring to FIG. 5B, the coil stent 50 has been cut from the coil stent blank and the coil stent remainder 92 remains in the sheath 66. The rotation of the helical screw 74 can be reversed to draw the coil stent remainder 92 into the sheath 66. The coil stent delivery system can then be removed from the vasculature.

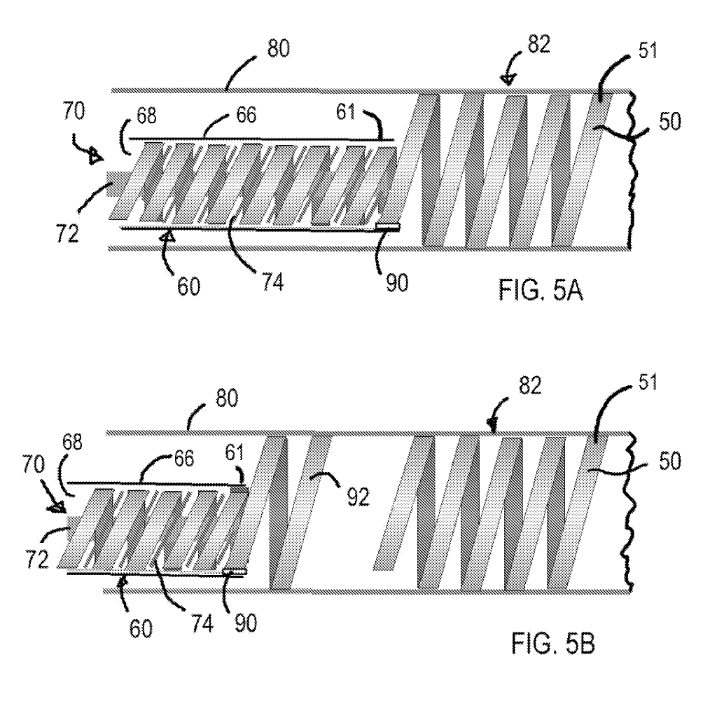

FIG. 6 is a cross section view of yet another embodiment of a coil stent delivery system made in accordance with the present invention. In this embodiment, the helical screw 174 includes screw teeth 192 oriented to engage the coil stent 50. The screw teeth 192 help grip the coil stent 50 and move it through the sheath 66.

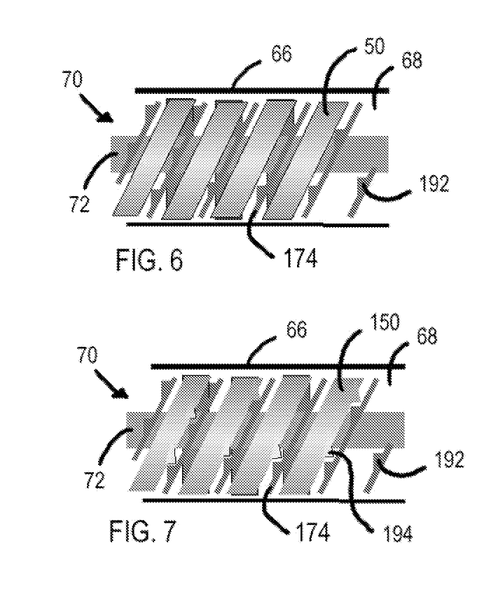

FIG. 7 is a cross section view of yet another embodiment of a coil stent delivery system made in accordance with the present invention. In this embodiment, the coil stent 150 has stent teeth 194 oriented to engage the screw teeth 192. The stent teeth 194 can help move the coil stent 150 through the sheath 66, anchor the coil stent 150 in the vessel, and/or prevent the coil stent 150 from slipping during deployment. The stent teeth 194 can be teeth and/or barbs as desired for a particular application.

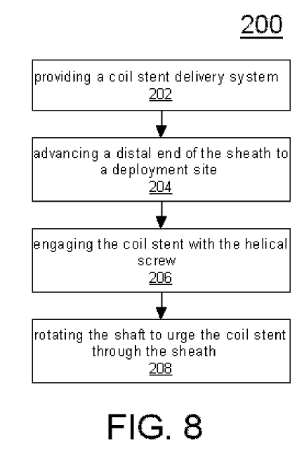

FIG. 8 is a flow chart for a method of delivering a coil stent in accordance with the present invention. The method 200 includes providing a coil stent delivery system 202, the coil stent delivery system having a sheath defining a sheath lumen and a shaft with a helical screw disposed about the shaft in the sheath lumen; advancing a distal end of the sheath to a deployment site 204; engaging the coil stent with the helical screw 206; and rotating the shaft 208 to rotate the helical screw and urge the coil stent through the sheath toward the deployment site. In one embodiment, the method 200 can further include rotating the shaft until the coil stent is free of the distal end of the sheath and deployed at the deployment site. In another embodiment, the method 200 can further include rotating the shaft until a portion of the coil stent is free of the distal end of the sheath, and retracting the sheath to deploy the coil stent at the deployment site.

In yet another embodiment, the method 200 can include rotating the shaft in a first direction until a portion of the coil stent is free of the distal end of the sheath; evaluating placement of the coil stent at the deployment site; and rotating the shaft in a second direction opposite the first direction to retract the portion of the stent into the sheath when the placement of the coil stent at the deployment site is not as desired. In yet another embodiment, the coil stent can be a coil stent blank, and the method 200 can include rotating the shaft until a desired length of the coil stent blank extends from the distal end of the sheath; and cutting the coil stent blank at the distal end of the sheath. In yet another embodiment, the method 200 can include counting rotations of the shaft to determine an axial location of the coil stent in the sheath.

It is important to note that FIGS. 1-8 illustrate specific applications and embodiments of the present invention, and are not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A stent delivery system comprising:
a coil stent;
a housing, the housing having a receiver defining a receiver chamber and a sheath defining a sheath lumen, the receiver chamber being in communication with the sheath lumen; and
a screw assembly, the screw assembly having a shaft, a helical screw disposed about a distal portion of the shaft, and a drive operably coupled to the shaft;
wherein the shaft is disposed in the receiver chamber and the sheath lumen, the helical screw is disposed in the sheath, and the coil stent is disposed about the shaft in the receiver chamber and engages the helical screw; and
wherein rotation of the drive moves the coil stent through the sheath lumen.

2. The stent delivery system of claim 1 wherein the coil stent has a relaxed diameter and a delivery diameter, the diameter of the receiver chamber being greater than or equal to the relaxed diameter, and the diameter of the sheath lumen being the delivery diameter.

3. The stent delivery system of claim 2 wherein the coil stent is sealed in the receiver chamber.

4. The stent delivery system of claim 1 wherein the coil stent is bioabsorbable.

5. The stent delivery system of claim 1 wherein at least an end portion of the coil stent is made of a super-elastic material.

6. The stent delivery system of claim 1 wherein the helical screw has screw teeth oriented to engage the coil stent.

7. The stent delivery system of claim 6 wherein the coil stent has stent teeth oriented to engage the screw teeth.

8. The stent delivery system of claim 1 wherein a cross section of the coil stent across a coil at a perimeter is selected from the group consisting of a circle, rectangle, and ellipse.

9. A delivery system for a coil stent comprising:
a housing, the housing having a receiver defining a receiver chamber and a sheath defining a sheath lumen, the receiver chamber being in communication with the sheath lumen; and
a screw assembly, the screw assembly having a shaft, a helical screw disposed about a distal portion of the shaft, and a drive operably coupled to the shaft;
wherein the shaft is disposed in the receiver chamber and the sheath lumen, the helical screw is disposed in the sheath, and the coil stent is disposed about the shaft in the receiver chamber and engages the helical screw; and
wherein rotation of the drive moves the coil stent through the sheath lumen.

10. The delivery system of claim 9 wherein the shaft defines a shaft lumen operable to receive a guidewire.

11. The delivery system of claim 9 wherein the shaft defines a shaft lumen and coolant disposed in the shaft lumen.

12. The delivery system of claim 9 wherein the shaft is a hypotube with grooves.

13. The delivery system of claim 12 wherein the hypotube includes more grooves in a distal portion of the shaft than in a proximal potion of the shaft.

14. The delivery system of claim 9 further comprising a low-tack adhesive disposed on the helical screw.

15. The delivery system of claim 9 wherein the drive is selected from the group consisting of a manual drive and a motor.

16. The delivery system of claim 9 further comprising a rotation counter operably connected to the helical screw.

17. The delivery system of claim 9 further comprising a cutter disposed at a distal end of the sheath.

18. A method of delivering a coil stent comprising:
providing a coil stent delivery system, the coil stent delivery system having a sheath defining a sheath lumen and a shaft with a helical screw disposed about the shaft in the sheath lumen;
advancing a distal end of the sheath to a deployment site;
engaging the coil stent with the helical screw; and
rotating the shaft to rotate the helical screw and urge the coil stent through the sheath toward the deployment site.

19. The method of claim 18 further comprising rotating the shaft until the coil stent is free of the distal end of the sheath and deployed at the deployment site.

20. The method of claim 18 further comprising:
rotating the shaft until a portion of the coil stent is free of the distal end of the sheath; and
retracting the sheath to deploy the coil stent at the deployment site.

21. The method of claim 18 further comprising:
rotating the shaft in a first direction until a portion of the coil stent is free of the distal end of the sheath;
evaluating placement of the coil stent at the deployment site; and
rotating the shaft in a second direction opposite the first direction to retract the portion of the stent into the sheath when the placement of the coil stent at the deployment site is not as desired.

22. The method of claim 18 wherein the coil stent is a coil stent blank, and the method further comprises:
rotating the shaft until a desired length of the coil stent blank extends from the distal end of the sheath; and
cutting the coil stent blank at the distal end of the sheath.

23. The method of claim 18 further comprising counting rotations of the shaft to determine an axial location of the coil stent in the sheath.
